(19) United States
(12) Patent Application Publication
(10) Pub. No.: US 2022/0058337 A1
(43) Pub. Date: Feb. 24, 2022

(54) GENERATING ORGANIC SYNTHESIS PROCEDURES FROM SIMPLIFIED MOLECULAR-INPUT LINE-ENTRY SYSTEM REACTION

(71) Applicant: INTERNATIONAL BUSINESS MACHINES CORPORATION, Armonk, NY (US)

(72) Inventors: Leonidas Georgopoulos, Zurich (CH); Joppe Geluykens, Zurich (CH); Alain Claude Vaucher, Zurich (CH); Philippe Schwaller, Duedingen (CH); Aleksandros Sobczyk, Ruschlikon (CH); Vishnu Harikrishnan Nair, Edappally (IN); Teodoro Laino, Rueschlikon (CH)

(21) Appl. No.: 16/995,853

(22) Filed: Aug. 18, 2020

(51) Int. Cl. G06F 40/205; G06F 40/56; G06K 9/62; G06N 20/00

(52) U.S. Cl. CPC G06F 40/205; G06F 40/56; G06K 9/6256; G06N 20/00; G06K 9/6263

(57) ABSTRACT

A computer-implemented method for generating an organic synthesis procedure from a simplified molecular-input line-entry system (SMILES) string may be provided. The method includes receiving a plurality of SMILES strings describing a desired chemical product and required reactants, and predicting procedure steps for an organic synthesis procedure for producing the desired chemical product by a deep machine-learning model system trained with sets of SMILES strings describing respective desired chemical products, reactants and related procedure steps as training data. The sets can be extracted from a corpus of associated chemical documents, and the predicted procedure steps are human readable. The method includes further receiving a modification signal for a modification to the predicting procedure steps, storing the plurality of received SMILES strings, the predicted procedure steps and the modification of the predicting procedure steps.

### BACKGROUND

The present invention relates generally to a method for generating an organic synthesis procedure, and more specifically, to a method for generating an organic synthesis procedure from a simplified molecular-input line-entry system, (hereinafter “SMILES”). The invention relates further to a system for generating an organic synthesis procedure from a simplified molecular-input line-entry system, and a computer program product.

### SUMMARY

According to one aspect of the present invention, a computer-implemented method for generating an organic synthesis procedure from a simplified molecular-input line-entry system, (hereinafter “SMILES”), string may be provided. The method may include receiving a plurality of SMILES strings of which a first portion describes a desired chemical product and a second remaining portion describes required reactants and predicting procedure steps for an organic synthesis procedure for producing the desired chemical product by a deep machine-learning model system trained with sets of SMILES strings describing respective desired chemical products, required reactants and related procedure steps as training data. Thereby, the sets may be extracted from a corpus of associated chemical documents. The predicted procedure steps may be human readable.

The method may further include receiving a modification signal for a modification to the predicting procedure steps, storing the received plurality of simplified molecular-input line-entry system strings, the predicted procedure steps and the modification to the predicting procedure steps.

According to another aspect of the present invention, a system for generating an organic synthesis procedure from a simplified molecular-input line-entry system string may be provided. The system may include first receiving means adapted for receiving a plurality of SMILES strings of which a first portion describes a desired chemical product and a second remaining portion describes required reactants and predicting procedure steps, and predicting means adapted for predicting procedure steps for an organic synthesis procedure for producing the desired chemical product by a deep machine-learning model system trained with sets of SMILES strings describing respective desired chemical products, required reactants and related procedure steps as training data. Thereby, the sets may be extracted from a corpus of associated chemical documents. The predicted procedure steps may be human readable.

The system may further include second receiving means adapted for receiving a modification signal for a modification to the predicted procedure steps, first storage means adapted for storing the plurality of received simplified molecular-input line-entry system string, the predicted procedure steps and the modification to the predicted procedure steps.

The proposed computer-implemented method—as well as the related system—for generating an organic synthesis procedure from a plurality of SMILES strings may offer multiple advantages, technical effects, contributions and/or improvements:

The described method and system may address a problem of generating a sequence of commands understandable by a human and a structured format and also understandable, potentially after a conversion, by a computer system linked to a chemical robot capable of executing these commands that may enable automating the process of executing a chemical synthesis and enabling human supervision at the same time.

This may be achieved by providing a plurality of molecules, in particular the reaction product and the required reactants, involved in the reaction in a common chemical format, e.g., SMILES, to an appropriate trained machine-learning model and then receiving a sequence of human understandable actions, i.e., procedure steps or recipes, required to perform the product the chemical product. The predicted actions may be presented to a human expert or supervisor for modification and validation and may then be stored as computer structured form art data, e.g., JSON (JavaScript Object Notation), YAML (“YAML Ain't Markup Language”), Protobuf (Protocol Buffers) serialization, in a data storage from where they may be picked up for execution by a robot array for chemical reactions to produce the requested chemical product.

A trained artificial intelligence system, i.e., the machine-learning (ML) system, may be instrumental in interpreting the SMILES strings into a possible and/or optimal sequence of processing steps based on the corpus, i.e., the body of chemical documents, of associated chemical documents, i.e., literature, in which combinations of SMILES strings describing specific chemical products and related and recommended procedural steps for the synthesis of the desired chemical product may also be described. The machine-learning system may be used to predict sequences of procedural steps even if a direct correspondence between a specific set of SMILES string and all required procedural steps are not described.

The sources for the training data for the ML system can be numerous. Chemical reaction procedures have been published for a long time in journals, text books, scientific articles, conference papers, via the Internet, private research databases as well as patent documents. The training data may be manually prepared for the ML system or may be gathered automatically, i.e., SMILES strings together with related reaction procedures. As examples, some sources may be named: the Beilstein database, the Reaxys database comprising more than 100 Mio. chemical compounds, 45 Mio. chemical reactions and 500 Mio. experimental facts, Scopus database by Elsevier, the ChemSpider database by the Royal Society of Chemistry, the SciFinder database by Chemical Abstract Service (CAS), the Science and Technical Information network (STN International by CAS), just to name a few. The Beilstein database is available through Reaxys®. Reaxys® is a registered trademark of Elsevier Properties SA.

However, even with the amount of information available to the modern scientist through chemical databases, a manual approach to synthesis, e.g., a new drug is impractical, is extremely time-consuming and expensive.

Therefore, the proposed method and the related system may represent an effective and elegant way to bridge the gap between the theoretically possible compound options and realistic experiment successes. To many researchers, this may be the only practical option to develop new drugs and/or chemical compounds (and other chemical compounds) given the above-discussed constraints.

Hence, this concept may enable higher productivity for wet-lab chemical experiments and using in an automatic fashion a large number of existing documents for producing the desired chemical products and at the same time reduce the number of required experiments. By the use of an additional analysis of the produced chemical product, a potential re-training of the machine-learning system and a successful repetition of the chemical experiment using—at least in parts human interventions—a closed-loop, self-optimizing system method and related system may be achievable.

In the following, additional embodiments of the inventive method, also applicable for the related system, will be described.

According to an extended embodiment, the method may also include converting the modified predicting procedure steps into a sequence of execution steps interpretable by a chemical robot, and storing the sequence of execution steps. This way, the method may support a more or less automatic production of the desired chemical product.

According to one advantageous embodiment, the method may also include executing the sequence of execution steps by the chemical robot. Thereby, the desired chemical product according to the first portion of the received SMILES string may be produced. This may largely bypass human interactions and entire process. Chemical experts may be released from routine tasks significantly.

According to permissive embodiments of the method, the first portion of the received simplified molecular-input line-entry system string may relate to at least one out of the group comprising polymers, polymer additives, catalysts, pesticides, dyes, fertilizers, artificial flavoring and sweeteners, compounds used in fundamental research, peptidomimetics, synthetic proteins, and nanostructures. Potentially, also other classes of chemical products may be addressed. Hence, a large number of chemical reaction products may be used in wet-lab experiments widely without human interventions.

According to one preferred embodiment, the method may also include a retraining of the deep machine-learning model system using sets of the SMILES strings and related modified predicted procedure steps. This way, the proposed method and related system may become better over time and may require less human effort for even more complex experimental situations.

According to a further useful embodiment, the method may also include extracting simplified molecular-input line-entry system strings from a text document. The text document may originate from a wide variety of different sources. For the analysis of the text document, regular expression analysis and/or natural language processing techniques may be used successfully.

According to one optional embodiment, the method may also include receiving the plurality of simplified molecular-input line-entry system string through a user interface or an application programming interface of the deep machine-learning model system. Hence, the proposed method does not restrict the source of a simplified molecular-input line-entry system string. Human originating , e.g., entered by a user interface, input may be accepted in the same way as input from another system.

According to one advantageous embodiment of the method, the reception of the modification signal may also include rendering the predicted procedure steps to a user interface and receive the modification signal indicative of a modification to the predicted procedure steps. Hence, human experience may be included as last expert resort before the wet-lab experiment may be conducted. This may save valuable resources in terms of time and material.

According to an advanced embodiment of the method, the chemical robot may be a first chemical robot, and wherein the sequence of execution steps are directed to the first chemical robot having first constraints, in particular, in the form of degrees of freedom for the potentially conducted experiments, the method may also include converting the sequence of execution steps for the first chemical robot to be executable by a second chemical robot underlying second constraints. Hence, the sequence of execution steps designed for a specific chemical robot, e.g., being made available as a library, may be converted to another chemical robot of another type with other constraints and capabilities in order to reflect technology advances and chemical robots. On the other side, if incompatibilities may exist between the experimental options of different chemical robots, an error message may be generated and an expert may manually modify the sequence of execution steps using a dialogue sub-system.

Consequently, and according to a further enhanced embodiment of the proposed method, the sequence of execution steps, interpretable by a first robot, may be converted automatically from the sequence of execution steps for the first robot to a sequence of execution steps interpretable by a second robot. Alternatively, the execution steps interpretable by the second chemical robot may also be derived from the sequence of procedure steps directly.

According to one additionally advanced embodiment, the method may also include analyzing, e.g., using a mass spectrometer, a product produced by executing the sequence of execution steps by the robot whether the desired chemical product has actually been produced. The method may also optionally allow modifying the procedure steps, using the above-described user interface, and retry the experiment in a semi-automated way.

According to another advantageous embodiment, the method may also include extending the training data set by the predicted procedure steps together with confidence factor value for a determination for a positive result that the desired chemical product has been produced, i.e., indicating that the performed procedure steps have been successful. The confidence factor value may be output to a user.

According to a further advantageous embodiment of the method, the training data may include as well technical constraint data of the chemical robot, i.e., basically describing the limitations in the options of the chemical robot. This may be performed using metadata being widely independent of a specific chemical robot. Thus, this aspect may also address limitations of chemical robots between different manufacturers.

Furthermore, embodiments may take the form of a related computer program product, accessible from a computer-usable or computer-readable medium providing program code for use, by, or in connection, with a computer or any instruction execution system. For the purpose of this description, a computer-usable or computer-readable medium may be any apparatus that may contain means for storing, communicating, propagating or transporting the program for use, by, or in connection, with the instruction execution system, apparatus, or device.

### BRIEF DESCRIPTION OF THE DRAWINGS

It should be noted that embodiments of the invention are described with reference to different subject-matters. In particular, some embodiments are described with reference to method type claims, whereas other embodiments are described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be disclosed within this document.

The aspects defined above, and further aspects of the present invention, are apparent from the examples of embodiments to be described hereinafter and are explained with reference to the examples of embodiments, but to which the invention is not limited.

Preferred embodiments of the invention will be described, by way of example only, and with reference to the following drawings:

FIG. 1 shows a block diagram of an embodiment of the inventive computer-implemented method for generating an organic simplified molecular-input line-entry system, (hereinafter “SMILES”), string;

FIG. 2 shows an exemplary flow of actions and involved components;

FIG. 3 shows a block diagram of an exemplary flow of activities starting with a SMILES string and ending with the procedure steps;

FIG. 4 shows an exemplary flowchart for an embodiment of the system for generating an organic synthesis procedure from a SMILES string;

FIG. 5 shows a block diagram for a computing system comprising the system according to FIG. 4;

FIG. 6 shows a cloud computing environment according to an embodiment of the present invention; and

FIG. 7 shows abstraction model layers according to an embodiment of the present invention.

### DETAILED DESCRIPTION

Performing chemical experiments for a synthesis of new molecules in the chemical, biochemical or pharmaceutical area is time-consuming and typically labor-intensive. In order to partially automate such synthesis experiments, chemical robots have been introduced allowing a software controlled experiment designed to by which a limited number of ingredients for the experiments may react in, e.g., a reaction chamber to produce a designed experiment output, instead of conducting such wet-lab experiments by humans. When the domain experts take advantage of automation hardware, they manage the complexity of programming the robot to execute specific tasks. These tasks are typically part of the domain knowledge of the experts and represent the decisive variable for successful chemical synthesis.

However, programming (partial-) autonomous chemical robotic hardware may represent a significant limitation, as the programming of these units may require highly skilled and proficient chemists with additional programming skills

In this context, some documents have been published for analyzing relationships between molecular structures and biological activities in one or more molecules by transforming molecular structured data into a hierarchical representation of chemical concepts and descriptors and detecting common tree-like patterns in the data and for designing and processing a rule pipeline for in silico predictions of chemical reactions.

However, also these approaches do not solve the problem of providing only the desired chemical reaction product molecule and leaving the details of how to conduct the experiment, in particular, the right individual procedure steps, to an experiment automation system because so far, too many human interventions may be required.

In the context of this description, the following conventions, terms and/or expressions may be used:

The term ‘simplified molecular-input line-entry system string’ (SMILES string) may denote the known specification in the form of a line notation for describing the structure of chemical species using short ASCII strings. SMILES strings may be imported by most molecule editors for conversion back into two-dimensional drawings or three-dimensional models of the molecules.

The term ‘organic synthesis procedure’ may denote a sequence of experimental steps in wet-lab chemistry for synthesizing a chemical product. It may be a step-wise described process that leads to the chemical transformation of one set of chemical substances to another. The substance (or substances), initially involved in a chemical reaction, are called reactants or reagents. Chemical reactions are usually characterized by a chemical change, and they yield one or more products, which usually have properties different from the reactants. Reactions often consist of a sequence of individual sub-steps, the so-called elementary reactions, and the information on the precise course of action is part of the reaction mechanism. Chemical reactions are described with chemical equations, which symbolically present the starting materials, end products, and sometimes intermediate products and reaction conditions. There are several notations for chemical equations and/or products, e.g., SMILES string.

The term ‘desired chemical product’ may denote the chemical product potentially be produced by the chemical robot and being describable by a SMILES string. A sequence of procedure steps may be required to arrive at the desired chemical product starting from a plurality of reactants.

The term ‘deep machine-learning (ML) model system’ may denote an artificial intelligence system using deep neural network comprising an input layer, an output layer and a plurality of embedded layers. Each layer may include a plurality of nodes receiving input typically from a plurality of nodes from a previous layer and delivering signals to a subsequent layer. Each node may perform—after training and in contrast to procedural programming—a specific transformation to the received signal(s) and adding a weighing function. The ML model may be trained with known data from which known output data shall be generated (supervised learning). The learning approach does not use procedural programming but may enable learning by example. This may be particularly useful when analyzing text documents to relate chemical substances (e.g., the here described desired chemical product) and procedure steps to arrive after a sequence of steps at the desired chemical product.

The term ‘modification to the predicted procedure steps’ may denote a manual intervention in the sequence of determined predicting procedure steps and converting these steps to a sequence of steps to be executable by the chemical robot. The human supervisor may alter the system-proposed procedure steps in order to avoid non-successful chemical experiments. At this point, the human experience may be combined with machine intelligence of the ML system.

The term ‘execution steps’ may denote individual steps performable and being performed by the chemical robot. The execution steps are not required to be machine readable; however, they may be useful instructions directly executed by the chemical robot.

The term ‘chemical robot’—also denoted as ‘laboratory robot’ ‘may denote a robot adapted to move biological or chemical samples around to synthesize novel chemical entities or to test a pharmaceutical value of existing chemical matter. Advanced laboratory robotics can be used to completely automate the process of science, as in the known robot science project. Laboratory processes are suited for robotic automation as the processes are composed of repetitive movements (e.g., pick/place, liquid & solid additions, heating/cooling, mixing, shaking, testing). Many laboratory robots are commonly referred to as auto-samplers, as their main task is to provide continuous samples for analytical devices.

The term ‘technical constraint data’ may denote a portion of a specification of a chemical robot. The constraints may, e.g., be defined as the maximum number of reagents, currently loaded reagents, available helper reagents (like water or a solvent) and temperature conditions selectable. However, the specifications of chemical robot show a large variety of limitations and options. Features describing these may here be interpreted as constraints.

In the following, a detailed description of the figures will be given. All instructions in the figures are schematic. Firstly, a block diagram of an embodiment of the inventive computer-implemented method for generating an organic synthesis procedure from a SMILES string is given. Afterwards, further embodiments, as well as embodiments of the related system, will be described.

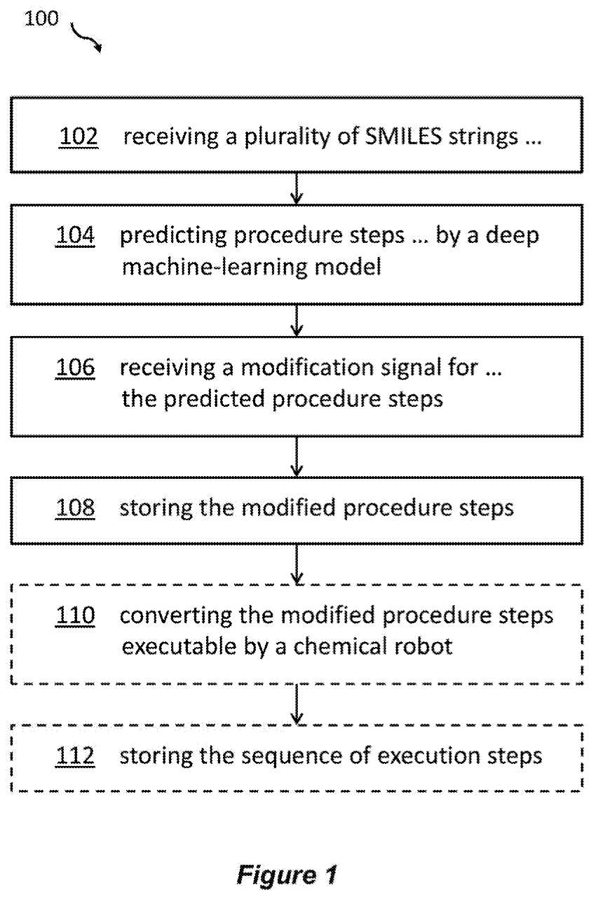

FIG. 1 shows a block diagram of a preferred embodiment of the computer-implemented method 100 for generating an organic synthesis procedure from a SMILES string, according to an embodiment.

The method 100 includes receiving, 102, a plurality of the SMILES strings of which a first portion describes a desired chemical product and a second remaining portion describes required reactants. The method 100 continues, predicting, 104, procedure steps for an organic synthesis procedure for producing the desired chemical product by a deep machine-learning (ML) model system. The deep ML model system has been trained with sets of SMILES strings describing respective desired chemical products, required reactants and related procedure steps as training data. This allows the sets to be extracted from a corpus of associated chemical documents, i.e., chemical literature. Any kind of the literature from libraries, scientific research, textbooks, and so on may be used. The predicted procedure steps are also human readable and may be displayed for a user.

Additionally, the method 100 includes receiving, 106, a modification signal for a modification of the predicted procedure steps. The method 100 continues, storing, 108, the received SMILES string, the predicted procedure steps and the modification of the predicted procedure steps. Optionally (marked with dashed lines), the method 100 also includes converting, 110, the modified predicted procedure steps into a sequence of execution steps interpretable by a chemical robot, and storing, 112, the sequence of execution steps, e.g. for later or immediate execution.

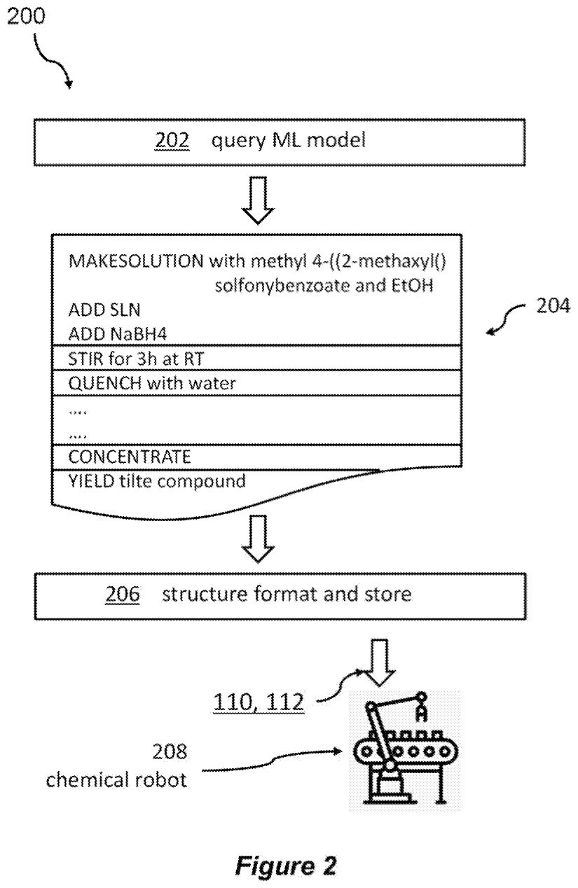

FIG. 2 shows an exemplary flow 200 of actions and involved components of the method in use, in an embodiment. After the system has received the chemical formula of the chemical reaction product and the reactants, the deep ML model is queried, 202. This may result in a list, 204, of procedure steps generated by the deep ML model system based on the previous training. It may be displayed (not shown) and potentially modified by a user and supervisor; then, 206, it may be converted in a machine-readable data structure format and stored to be used by the chemical robot 206. The proposed system is not part (but can be) of the chemical robot 208. The machine readable form of execution steps (conversion symbolized by 110, 112) can optionally be transmitted to the chemical robot 208. The chemical robot may also be positioned in a remote location—e.g., in a protected area in order to bring the supervisor in danger—if compared to the proposed system.

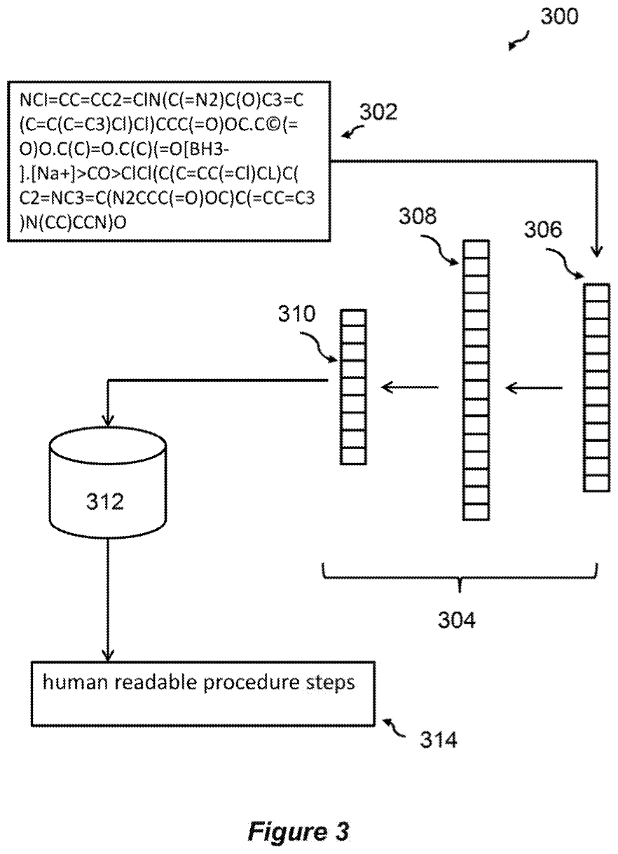

FIG. 3 shows a block diagram of an exemplary flow 300 of activities starting with concatenated SMILES strings 302 and ending with procedure steps 314, according to an embodiment. The SMILES strings 302 (an exemplary concatenation of SMILES strings is shown) is used as input for the deep ML model system 304. It may be directed to the input layer 306 of the deep ML model system 304. It may be embedded by a plurality of hidden layers 308 in the respective ML system model vector space. The output layer 310 of the ML model system 304 may have a pointer to a recipe to produce the desired chemical product in form of a list of procedure steps 314 which are human readable. These may be stored in data storage 312.

Additionally, these procedure steps 314 may be displayed to the user/supervisor/operator for potential modifications before the procedure steps 314 may be converted to a sequence of execution steps interpretable by the chemical robot (not shown).

The following table 1 shows exemplary procedure steps:

TABLE 1

| | |
|---|---|
| 1. | MAKESOLUTION with methyl 3-7-amoni-2- |
| | [(2,4-diclorophenyl)(hydroxy)methyl]- |
| | 1H-benzinmidazol -1-yYIpropanoute (6.00 g, 14,7 mmol) |
| | and methanol (147 mL); |
| 2. | ADD SLN |
| 3. | ADD accetalhyde (4.95 mL, 88.2 mmol) at 0° C.; |
| 4. | WAIT 30 min; |
| 5. | ADD sodium acetoxyborohydride (18.7 g, 88.2 mmol) |
| 6. | WAIT 2 h; |
| 7. | QUENCH with water; |
| 8. | CONCENTRATE; |
| 9. | ADD ethyl acetate; |
| 10. | WASH with sodium hydroxide (1M); |
| 11. | WASH with brine; |
| 12. | DRYWITHMATERIAL sodium sulfate; |
| 13. | FILTER keep filtrate; |
| 14. | CONCENTRATE; |
| 15. | PURIFY; |
| 16. | YIELD title compound (6.30 g, 13.6 mmol, 92%) |

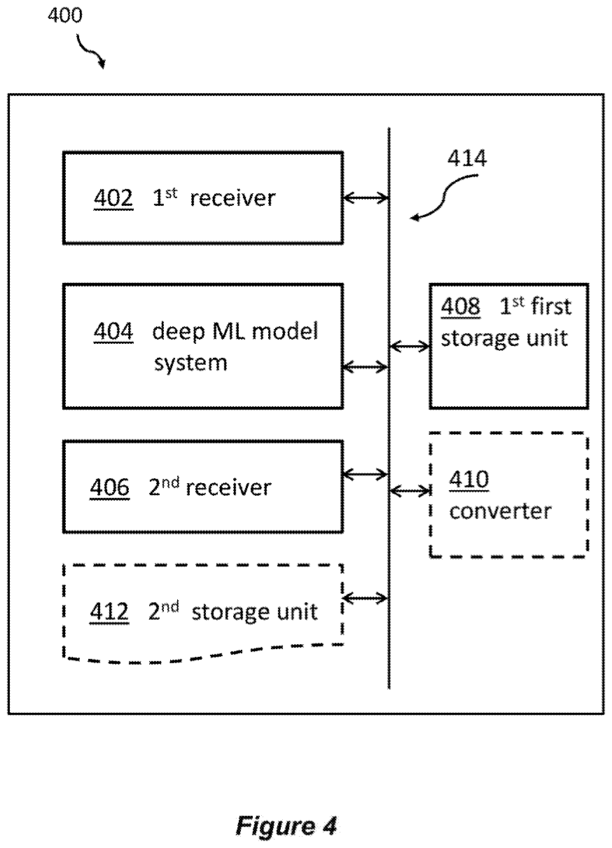

FIG. 4 shows an exemplary flowchart for an embodiment of the system 400 for generating an organic synthesis procedure from a SMILES string, according to an embodiment. The system 400 includes first receiving means, in particular, a first receiver 402, adapted for receiving the SMILES string describing a desired chemical product and predicting means, in particular, a deep machine-learning model system 404, adapted for predicted procedure steps for an organic synthesis procedure for producing the desired chemical product. The deep ML model-based system is trained with sets of simplified SMILES strings describing respective desired chemical products, required reactants and related procedure steps as training data, wherein the sets are extracted from a corpus of associated chemical documents. The predicted procedure steps are human readable.

The system 400 also includes second receiving means, in particular, a second receiver 406, adapted for receiving a modification signal for a modification to the predicted procedure steps. The system 400 includes a first storage means, in particular, a first storage unit 408, adapted for storing the received SMILES string, the predicted procedure steps and the modification of the predicted procedure steps.

The system 400 includes a conversion means, in particular, converter 410, adapted for converting the modified predicted procedure steps into a sequence of execution steps interpretable by a chemical robot. The system 400 includes a second storage means, in particular, second storage unit 412, adapted for storing the sequence of execution steps. The first and the second storage unit 408, 412, may be the same with a related storage management system.

It may be noted that the mentioned units and modules may be interconnected directly or indirectly for an information and/or signal exchange. Alternatively, the first receiver 402, the deep ML model-based system 404, the first storage unit 408, and optionally (marked with dashed lines) the converter 410, and the second stage unit 412 may be interconnected via the system internal bus system 414.

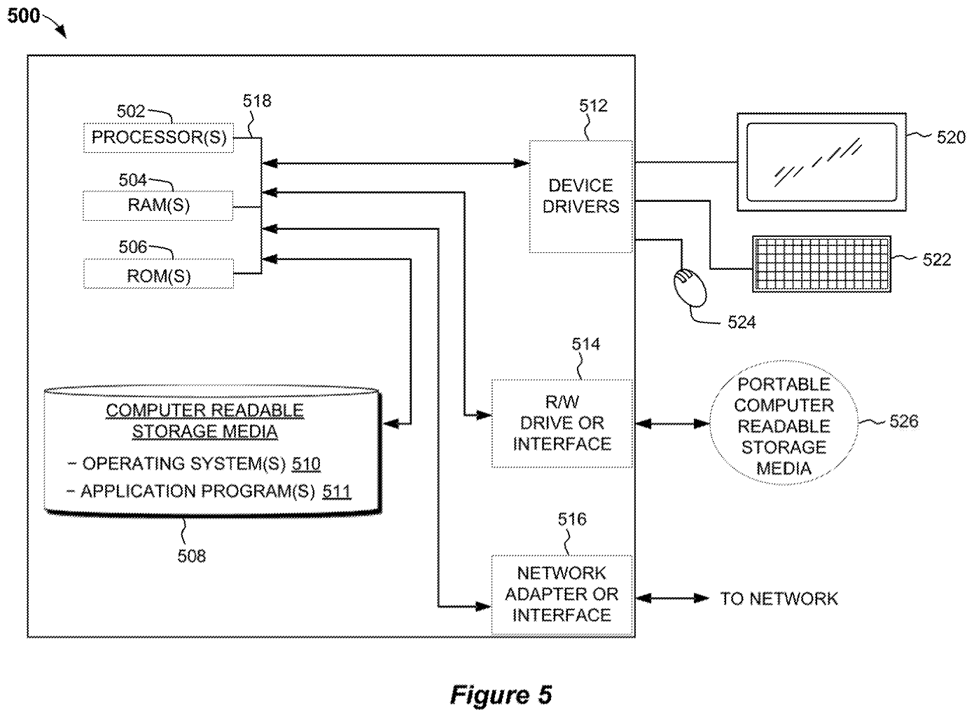

Embodiments of the invention may be implemented together with virtually any type of computer, regardless of the platform being suitable for storing and/or executing program code. FIG. 5 shows, as an example, a computing device 500 suitable for executing program code related to the proposed method.

The computing device 500 is only one example of a suitable computer system, and is not intended to suggest any limitation as to the scope of use or functionality of embodiments of the invention described herein, regardless, whether the computing device 500 is capable of being implemented and/or performing any of the functionality set forth hereinabove. In the computing device 500, there are components, which are operational with numerous other general purpose or special purpose computing system environments or configurations. Examples of well-known computing systems, environments, and/or configurations that may be suitable for use with the computing device 500 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like. The computing device 500 may be described in the general context of computer system-executable instructions, such as program modules, being executed by a computing device 500. Generally, program modules may include routines, programs, objects, components, logic, data structures, and so on that perform particular tasks or implement particular abstract data types. The computing device 500 may be practiced in distributed cloud computing environments where tasks are performed by remote processing devices that are linked through a communications network. In a distributed cloud computing environment, program modules may be located in both, local and remote computer system storage media, including memory storage devices.

As shown in FIG. 5, a block diagram of components of a computing device, in accordance with an embodiment of the present invention is shown. It should be appreciated that FIG. 5 provides only an illustration of an implementation and does not imply any limitations with regard to the environments in which different embodiments may be implemented. Many modifications to the depicted environment may be made.

The computing device may include one or more processors 502, one or more computer-readable RAMs 504, one or more computer-readable ROMs 506, one or more computer readable storage media 508, device drivers 512, read/write drive or interface 514, network adapter or interface 516, all interconnected over a communications fabric 518. Communications fabric 518 may be implemented with any architecture designed for passing data and/or control information between processors (such as microprocessors, communications and network processors, etc.), system memory, peripheral devices, and any other hardware components within a system.

One or more operating systems 510, and one or more application programs 511 are stored on one or more of the computer readable storage media 508 for execution by one or more of the processors 502 via one or more of the respective RAMs 504 (which typically include cache memory). For example, the exemplary flow 200 and the exemplary flow 300, may each be stored on the one or more of the computer readable storage media 508. In the illustrated embodiment, each of the computer readable storage media 508 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

The computing device may also include the R/W drive or interface 514 to read from and write to one or more portable computer readable storage media 526. Application programs 511 on the computing device may be stored on one or more of the portable computer readable storage media 526, read via the respective R/W drive or interface 514 and loaded into the respective computer readable storage media 508.

The computing device may also include the network adapter or interface 516, such as a TCP/IP adapter card or wireless communication adapter (such as a 4G wireless communication adapter using OFDMA technology). Application programs 511 may be downloaded to the computing device from an external computer or external storage device via a network (for example, the Internet, a local area network or other wide area network or wireless network) and network adapter or interface 516. From the network adapter or interface 516, the programs may be loaded onto computer readable storage media 508. The network may comprise copper wires, optical fibers, wireless transmission, routers, firewalls, switches, gateway computers and/or edge servers.

The computing device may also include a display screen 520, a keyboard or keypad 522, and a computer mouse or touchpad 524. Device drivers 512 interface to display screen 520 for imaging, to keyboard or keypad 522, to computer mouse or touchpad 524, and/or to display screen 520 for pressure sensing of alphanumeric character entry and user selections. The device drivers 512, R/W drive or interface 514 and network adapter or interface 516 may comprise hardware and software (stored on computer readable storage media 508 and/or ROM 506).

Additionally, the system 400 for generating an organic synthesis procedure from a SMILES string may be attached to the communications fabric 518.

The descriptions of the various embodiments of the present invention have been presented for purposes of illustration, but are not intended to be exhaustive or limited to the embodiments disclosed. Many modifications and variations will be apparent to those of ordinary skills in the art without departing from the scope and spirit of the described embodiments. The terminology used herein was chosen to best explain the principles of the embodiments, the practical application or technical improvement over technologies found in the marketplace, or to enable others of ordinary skills in the art to understand the embodiments disclosed herein.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access a normalized search engine or related data available in the cloud. For example, the normalized search engine could execute on a computing system in the cloud and execute normalized searches. In such a case, the normalized search engine could normalize a corpus of information and store an index of the normalizations at a storage location in the cloud. Doing so allows a user to access this information from any computing system attached to a network connected to the cloud (e.g., the Internet).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

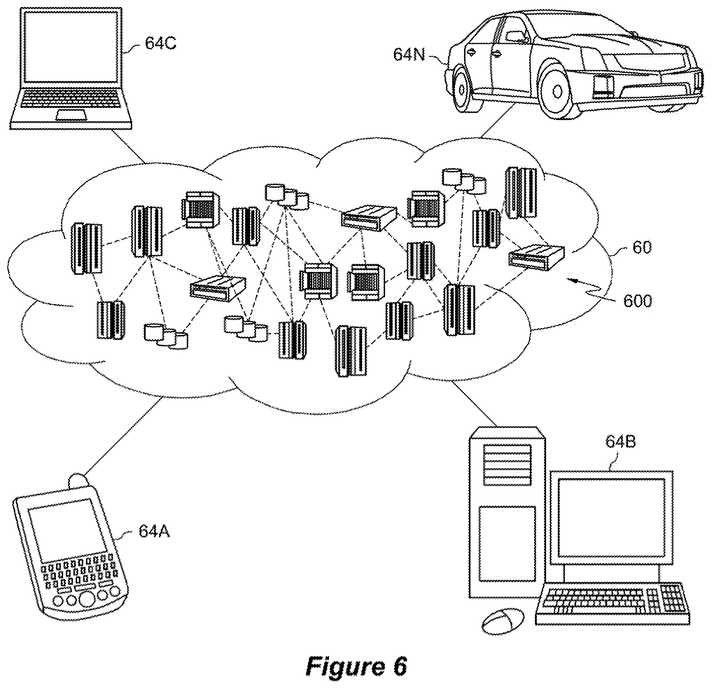

Referring now to FIG. 6, illustrative cloud computing environment 600 is depicted. As shown, cloud computing environment 600 includes one or more cloud computing nodes 610 with which local computing devices used by cloud consumers, such as, for example, personal digital assistant (PDA) or cellular telephone 640A, desktop computer 640B, laptop computer 640C, and/or automobile computer system 640N may communicate. Cloud computing nodes 610 may communicate with one another. They may be grouped (not shown) physically or virtually, in one or more networks, such as Private, Community, Public, or Hybrid clouds as described hereinabove, or a combination thereof. This allows cloud computing environment 600 to offer infrastructure, platforms and/or software as services for which a cloud consumer does not need to maintain resources on a local computing device. It is understood that the types of computing devices 640A-N shown in FIG. 6 are intended to be illustrative only and that cloud computing nodes 610 and cloud computing environment 600 can communicate with any type of computerized device over any type of network and/or network addressable connection (e.g., using a web browser).

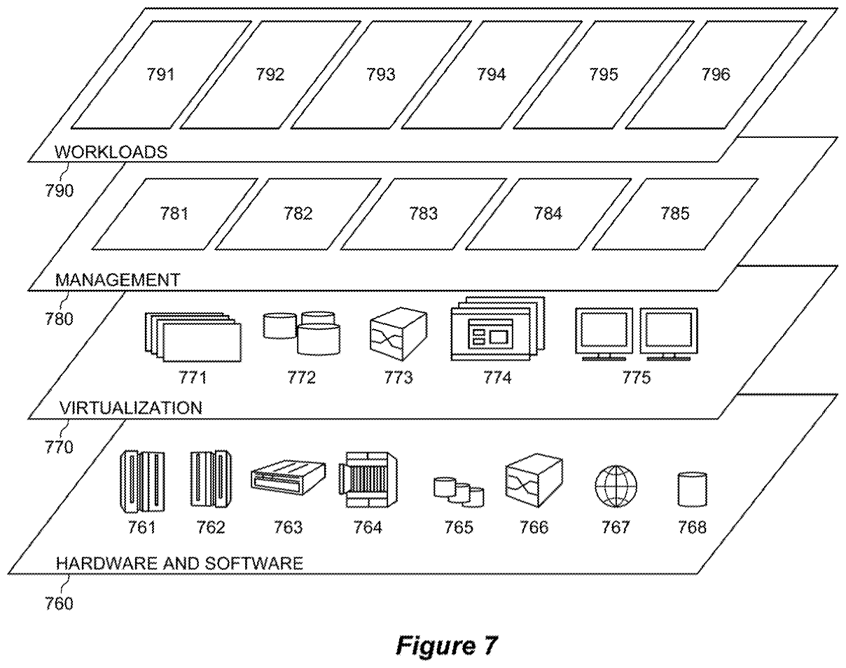

Referring now to FIG. 7, a set of functional abstraction layers provided by cloud computing environment 600 (as shown in FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 760 includes hardware and software components. Examples of hardware components include: mainframes 761; RISC (Reduced Instruction Set Computer) architecture based servers 762; servers 763; blade servers 764; storage devices 765; and networks and networking components 766. In some embodiments, software components include network application server software 767 and database software 768.

Virtualization layer 770 provides an abstraction layer from which the following examples of virtual entities may be provided: virtual servers 771; virtual storage 772, for example the one or more computer readable storage media 508 as shown in FIG. 5; virtual networks 773, including virtual private networks; virtual applications and operating systems 774; and virtual clients 775.

In an example, management layer 780 may provide the functions described below. Resource provisioning 781 provides dynamic procurement of computing resources and other resources that are utilized to perform tasks within the cloud computing environment. Metering and Pricing 782 provide cost tracking as resources are utilized within the cloud computing environment, and billing or invoicing for consumption of these resources. In an example, these resources may include application software licenses. Security provides identity verification for cloud consumers and tasks, as well as protection for data and other resources. User portal 783 provides access to the cloud computing environment for consumers and system administrators. Service level management 784 provides cloud computing resource allocation and management such that required service levels are met. Service Level Agreement (SLA) planning and fulfillment 685 provide pre-arrangement for, and procurement of, cloud computing resources for which a future requirement is anticipated in accordance with an SLA.

Workloads layer 790 provides examples of functionality for which the cloud computing environment may be utilized. Examples of workloads and functions which may be provided from this layer include: mapping and navigation 791; software development and lifecycle management 792; virtual classroom education delivery 793; data analytics processing 794; transaction processing 795; and generation of an organic synthesis program 796. The generation of an organic synthesis program 796 may generate an organic synthesis procedure.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

In a nutshell, die inventive concept may be summarized by the following clauses:
1. A method for generating an organic synthesis procedure from a simplified molecular-input line-entry system string, the method comprising
receiving a plurality of the simplified molecular-input line-entry system strings of which a first portion describes a desired chemical product and a second remaining portion describes required reactants,
predicting procedure steps for an organic synthesis procedure for producing the desired chemical product by a deep machine-learning model system trained with sets of simplified molecular-input line-entry system strings describing respective desired chemical products, required reactants and related procedure steps as training data, wherein the sets are extracted from a corpus of associated chemical documents, wherein the predicted procedure steps are human readable,
receiving a modification signal for a modification of the predicting procedure steps, and
storing the received plurality of simplified molecular-input line-entry system strings, the predicted procedure steps and the modification to the predicting procedure steps.
2. The method according to clause 1, also comprising
converting the modified predicting procedure steps into a sequence of execution steps interpretable by a chemical robot, and
storing the sequence of execution steps.
3. The method according to clause 1 or 2, also comprising
executing the sequence of execution steps by the chemical robot, thereby producing the desired chemical product according to the first portion of the received simplified molecular-input line-entry system strings.
4. The method according to any of the preceding clauses, wherein the first portion of the plurality of received simplified molecular-input line-entry system string relates to at least one out of the group comprising polymers, polymer additives, catalysts, pesticides, dyes, fertilizers, artificial flavouring and sweeteners, compounds used in fundamental research, peptidomimetics, synthetic proteins, and nanostructures for generating an organic synthesis procedure from a simplified molecular-input line-entry system (SMILES) string.
5. The method according to any of the preceding clauses, also comprising
retraining of the deep machine-learning model system using sets of the simplified molecular-input line-entry system strings and related modified predicted procedure steps.
6. The method according to any of the preceding clauses, also comprising
extracting the plurality of simplified molecular-input line-entry system strings from a text document.
7. The method according to any of the preceding clauses, also comprising
receiving the plurality of simplified molecular-input line-entry system strings through a user interface or an application programming interface of the deep machine-learning model system.
8. The method according to any of the preceding clauses, wherein the reception of the modification signal also comprises
rendering the predicted procedure steps to a user interface and receive the modification signal indicative of a modification to the predicted procedure steps.
9. The method according to any of the preceding clauses, wherein the chemical robot is a first chemical robot and wherein the sequence of execution steps are directed to the first chemical robot having first constraints, the method also comprising
converting the sequence of execution steps for the first chemical robot to be executable by a second chemical robot underlying second constraints.
10. The method according to any of the preceding clauses, wherein the sequence of execution steps is interpretable by a first robot and wherein the method comprises
converting the sequence of execution steps for the first robot to a sequence of execution steps interpretable by a second robot.
11. The method according to any of the preceding clauses, also comprising
analysing a product produced by executing the sequence of execution steps by the robot whether the desired chemical product was produced.
12. The method according to clause 11, also comprising
upon a determination that the desired chemical product has been produced, extending the training data set by the predicted procedure steps together with a confidence factor value.
13. The method according to any of the preceding clauses, wherein the training data comprise as well technical constraint data of the chemical robot.
14. A system for generating an organic synthesis procedure from a simplified molecular-input line-entry system string, the system comprising
first receiving means adapted for receiving a plurality of the simplified molecular-input line-entry system strings of which a first portion describes a desired chemical product and a second remaining portion describes required reactants,
predicting means adapted for predicting procedure steps for an organic synthesis procedure for producing the desired chemical product by a deep machine-learning model system trained with sets of simplified molecular-input line-entry system strings describing respective desired chemical products, required reactants and related procedure steps as training data, wherein the sets are extracted from a corpus of associated chemical documents, wherein the predicted procedure steps are human readable,
second receiving means adapted for receiving a modification signal for a modification of the predicting procedure steps, and
first storage means adapted for storing the plurality of received simplified molecular-input line-entry system strings, the predicted procedure steps and the modification of the predicting procedure steps.
15. The system according to clause 14, also comprising
execution means adapted for executing the sequence of execution steps by the chemical robot, thereby producing the desired chemical product according to the first portion of the received simplified molecular-input line-entry system strings.
16. The system according to clause 14 or 15, wherein the first portion of received simplified molecular-input line-entry system strings relates to at least one out of the group comprising polymers, polymer additives, catalysts, pesticides, dyes, fertilizers, artificial flavouring and sweeteners, compounds used in fundamental research, peptidomimetics, synthetic proteins, and nanostructures.
17. The system according to any of the clauses 14 to 16, also comprising
retraining means adapted for retraining of the deep machine-learning model system using sets of the simplified molecular-input line-entry system strings, the required reactants and related modified predicted procedure steps.
18. The system according to any of the clauses 14 to 17, also comprising
extracting means adapted for extracting the plurality of simplified molecular-input line-entry system strings from a text document.
19. The system according to any of the clauses 14 to 18, also comprising
third receiving means receiving the plurality of simplified molecular-input line-entry system strings through a user interface or an application programming interface of the deep machine-learning model system.
20. The system according to any of the clauses 14 to 19, wherein the reception of the modification signal also comprises
rendering means adapted for rendering the predicted procedure steps to a user interface and receive the modification signal indicative of a modification to the predicted procedure steps.
21. The system according to any of the clauses 14 to 20, wherein the chemical robot is a first chemical robot and wherein the sequence of execution steps are directed to the first chemical robot having first constraints, the system also comprising
converting means adapted for converting the sequence of execution steps for the first chemical robot to be executable by a second chemical robot underlying second constraints.
22. The system according to any of the clauses 14 to 21, wherein the sequence of execution steps are interpretable by a first robot, the system also comprising
converting means adapted converting the sequence of execution steps for the first robot to a sequence of execution steps interpretable by a second robot.
23. The system according to any of the clauses 14 to 22, also comprising
analysing means adapted for analysing a product produced by executing the sequence of execution steps by the robot whether the desired chemical product was produced.
24. The system according to clause 23, also comprising
extending means adapted for extending the training data set by the predicted procedure steps together with confidence factor for a determination that the desired chemical product has been produced.
25. A computer program product for generating an organic synthesis procedure from a simplified molecular-input line-entry system string, the program instructions being executable by one or more computing systems or controllers to cause the one or more computing systems to,
receive a plurality of the simplified molecular-input line-entry system strings of which a first portion describes a desired chemical product and a second remaining portion describes required reactants,
predict procedure steps for an organic synthesis procedure for producing the desired chemical product by a deep machine-learning model system trained with sets of simplified molecular-input line-entry system strings describing respective desired chemical products, required reactants and related procedure steps as training data, wherein the sets are extracted from a corpus of associated chemical documents, wherein the predicting procedure steps are human readable,
receive a modification signal for a modification of the predicted procedure steps, and
store the received simplified molecular-input line-entry system string, the predicted procedure steps and the modification of the predicting procedure steps.

What is claimed is:

1. A method for generating an organic synthesis procedure from a simplified molecular-input line-entry system (SMILES) string, the method comprising:
receiving a plurality of the SMILES strings wherein a first portion describes a desired chemical product and a second remaining portion describes required reactants;
predicting procedure steps for an organic synthesis procedure for producing the desired chemical product by a deep machine-learning model system trained with sets of SMILES strings describing respective desired chemical products, required reactants and related procedure steps as training data, wherein the sets are extracted from a corpus of associated chemical documents, wherein the predicted procedure steps are human readable;
receiving a modification signal for a modification of the predicting procedure steps; and
storing the received plurality of SMILES strings, the predicted procedure steps and the modification to the predicting procedure steps.

2. The method according to claim 1, further comprising:
converting the modified predicting procedure steps into a sequence of execution steps interpretable by a chemical robot; and
storing the sequence of execution steps.

3. The method according to claim 2, further comprising:
executing the sequence of execution steps by the chemical robot, thereby producing the desired chemical product according to the first portion of the received SMILES strings.

4. The method according to claim 1, wherein the first portion of the plurality of received SMILES strings relates to at least one out of a group comprising polymers, polymer additives, catalysts, pesticides, dyes, fertilizers, artificial flavouring and sweeteners, compounds used in fundamental research, peptidomimetics, synthetic proteins, and nanostructures for generating an organic synthesis procedure from a SMILES string.

5. The method according to claim 1, further comprising:
retraining of the deep machine-learning model system using sets of the SMILES strings and related modified predicted procedure steps.

6. The method according to claim 1, further comprising:
extracting the plurality of SMILES strings from a text document.

7. The method according to claim 1, further comprising:
receiving the plurality of SMILES strings through a user interface or an application programming interface of the deep machine-learning model system.

8. The method according to claim 1, wherein the reception of the modification signal further comprises:
rendering the predicted procedure steps to a user interface and receive the modification signal indicative of a modification to the predicted procedure steps.

9. The method according to claim 2, wherein the chemical robot is a first chemical robot and wherein the sequence of execution steps are directed to the first chemical robot having first constraints, and wherein the method further comprising converting the sequence of execution steps for the first chemical robot to be executable by a second chemical robot underlying second constraints.

10. The method according to claim 2, wherein the sequence of execution steps is interpretable by a first robot and wherein the method further comprises converting the sequence of execution steps for the first robot to the sequence of execution steps interpretable by a second robot.

11. The method according to claim 2, further comprising:
analysing a product produced by executing the sequence of execution steps by the robot whether the desired chemical product was produced.

12. The method according to claim 10, further comprising:
upon a determination that the desired chemical product has been produced, extending the training data by the predicted procedure steps together with a confidence factor value.

13. The method according to claim 1, wherein the training data further comprises technical constraint data of a chemical robot.

14. A system for generating an organic synthesis procedure from a simplified molecular-input line-entry system (SMILES) string, the system comprising:
a first receiving means adapted for receiving a plurality of the SMILES strings of which a first portion describes a desired chemical product and a second remaining portion describes required reactants;
a predicting means adapted for predicting procedure steps for an organic synthesis procedure for producing the desired chemical product by a deep machine-learning model system trained with sets of SMILES strings describing respective desired chemical products, required reactants and related procedure steps as training data, wherein the sets are extracted from a corpus of associated chemical documents, wherein the predicted procedure steps are human readable;
a second receiving means adapted for receiving a modification signal for a modification of the predicting procedure steps; and
a first storage means adapted for storing the plurality of received SMILES strings, the predicted procedure steps and the modification of the predicting procedure steps.

15. The system according to claim 14, further comprising:
an execution means adapted for executing the sequence of execution steps by a chemical robot, thereby producing the desired chemical product according to the first portion of the received SMILES strings.

16. The system according to claim 14, wherein the first portion of received SMILES strings relates to at least one out of a group comprising polymers, polymer additives, catalysts, pesticides, dyes, fertilizers, artificial flavouring and sweeteners, compounds used in fundamental research, peptidomimetics, synthetic proteins, and nanostructures.

17. The system according to claim 14, further comprising:
a retraining means adapted for retraining of the deep machine-learning model system using sets of the SMILES strings, the required reactants and related modified predicted procedure steps.

18. The system according to claim 14, further comprising:
an extracting means adapted for extracting the plurality of SMILES strings from a text document.

19. The system according to claim 14, further comprising:
a third receiving means receiving the plurality of SMILES strings through a user interface or an application programming interface of the deep machine-learning model system.

20. The system according to claim 14, wherein the reception of the modification signal further comprises:
a rendering means adapted for rendering the predicted procedure steps to a user interface and receive the modification signal indicative of a modification to the predicted procedure steps.

21. The system according to claim 15 wherein the chemical robot is a first chemical robot and wherein the sequence of execution steps are directed to the first chemical robot having first constraints, the system also comprising a converting means adapted for converting the sequence of execution steps for the first chemical robot to be executable by a second chemical robot underlying second constraints.

22. The system according to claim 15, wherein the sequence of execution steps are interpretable by a first robot, the system also comprising a converting means adapted converting a sequence of execution steps for the first robot to the sequence of execution steps interpretable by a second robot.

23. The system according to claim 15, also comprising an analysing means adapted for analysing a product produced by executing the sequence of execution steps by the robot whether the desired chemical product was produced.

24. The system according to claim 23, also comprising an extending means adapted for extending the training data by the predicted procedure steps together with confidence factor for a determination that the desired chemical product has been produced.

25. A computer program product for generating an organic synthesis procedure from a simplified molecular-input line-entry system (SMILES) string, the computer program product comprising:
one or more computer-readable tangible storage medium and program instructions stored on at least one of the one or more tangible storage medium, the program instructions executable by a processor, the program instructions being executable to:
receive a plurality of the SMILES strings of which a first portion describes a desired chemical product and a second remaining portion describes required reactants;
predict procedure steps for an organic synthesis procedure for producing the desired chemical product by a deep machine-learning model system trained with sets of SMILES strings describing respective desired chemical products, required reactants and related procedure steps as training data, wherein the sets are extracted from a corpus of associated chemical documents, wherein the predicting procedure steps are human readable;
receive a modification signal for a modification of the predicted procedure steps; and
store the received SMILES string, the predicted procedure steps and the modification of the predicting procedure steps.
